(19) United States
(12) Patent Application Publication
(10) Pub. No.: US 2007/0071706 A1
(43) Pub. Date: Mar. 29, 2007

(54) Lipodissolving dermatological topical preparation

(76) Inventors: Filiberto Zadini, North Hills, CA (US); Giorgio Zadini, Camarillo, CA (US)

Correspondence Address: FILIBERTO ZADINI, 2237 HILLTOP LANE, CAMARILLO, CA, 93012, US

(21) Appl. No.: 11/525,433

(22) Filed: Sep. 22, 2006

Related U.S. Application Data
(60) Provisional application No. 60/721,228, filed on Sep. 28, 2005.

(51) Int. Cl. A61K 8/73

(52) U.S. Cl. 424/70.13

(57) ABSTRACT

A dermatological topical preparation such as a cream, a lotion, an emulsion, a paste, an ointment and the likes where a lipo-dissolving emulsifier such as a biliary compound is transdermally delivered through the superficial layers of the skin into the subcutaneous adipose tissue with the use of skin permeability enhancers. Some of these skin permeability enhancers are cyclodextrins, while others are substances which have the peculiar property of enhancing delivery of the lipo-dissolving substance locally into the adipose subcutaneous tissue maximizing subcutaneous uptake while minimizing systemic absorption in order to achieve maximization of local effect.

### RELATED CASES

This Patent Application corresponds to Applicants Provisional Patent Application No. 60/721,228 entitled “Lipodissolving Dermatological Topical Preparation” filed on Sep. 28, 2005.

### FIELD OF THE INVENTION

The invention consists of a topical dermatological preparation containing a lipo-dissolving or adipocytes disrupting/lytic substance for treatment of localized adiposity.

### BACKGROUND—DESCRIPTION OF THE PRIOR ART

Numerous non-surgical treatments, topical and non topical, are available today for treatment of localized adiposities and lipodystrophies. Some of these treatments have a scientific base, some a pseudo-scientific, empiric base.

Among the topical treatments, only the treatments delivered via intradermal injections of lipolytic compounds have achieved clinically satisfactory results.

Topical treatments based on transdermal delivery of medications using methods other than percutaneous injections have high rate of failure. Indeed trssdermal delivery of lipo-dissolving preparation via the use of topical conventional preparations such as creams, lotions, emulsions, pastes, ointments is destined to failure as only a very small percentage, if any, of such lipo-dissolving preparations can get thru the nearly impermeable barrier of the horny impermeable layer of the skin known as stratum corneum.

Due to the above mentioned physiological obstacle to deep penetration represented by the stratum comeum, chemical compounds that have shown lipo-dissolving activity when introduced via injections, have consistently failed to reproduce their lipo-dissolving activity when incorporated in conventional creams, lotions, emulsions, pastes, ointments and the likes.

### BRIEF SUMMARY OF THE INVENTION

With the present invention Applicants propose a topical dermatological preparation having the characteristics of being capable of delivering percutaneously into subcutaneous adipose tissue or its proximity, chemical compounds such as a biliary acid or salt via the utilization of specific skin penetration enhancers. Biliary compounds have shown to effectively dissolve fat deposits when introduced percutaneously via injection into the fat deposits and or their proximity.

More specifically, the present invention discloses a topical preparation which utilizes a variety of skin penetration enhancers to enhance penetration and delivery into the subcutaneous adipose tissue of a proven lipodissolving compound. In the present invention the transdermal delivery of a biliary compound such as the deoxycholic acid or its salts or derivatives or precursors or other chemicals with detergent effects is aided by a skin penetration enhancer which enhances the passage of the biliary compound thru the skin barrier and facilitates its delivery into the subcutaneous target adipose tissue.

Applicants believe that cyclodextrins are a preferred skin penetration enhancers due to their physical-chemical characteristics. Applicants disclose however a variety of skin penetration enhancers that have historically proven to be able to aid the delivery of drugs and or cosmetics/cosmoceuticals thru the skin.

Applicants also disclose the use of lipo-issolving agents with a mixture of three components, Benzyl Alcohol, Acetone and Isopropanol, such a mixture being used as transdernal permeability enhancer. When used as a transdermal permeability enhancer, as documented in a scientific study conducted at USC cited in the Specifications, this mixture has shown to produce significant and persistent local accumulation in the subcutaneous tissue of the active ingredient being trnnsdermally delivered, combined with a negligible systemic absorption of such active ingredient, with resulting maximization of the local effect of the active ingredient. This mixture represents an ideal type of transdermal permeability enhancer for substances aimed at exhibiting their pharmacological effect locally in the subcutaneous tissue, such as for substances aimed at dissolving the subcutaneous adipose tissue.

### OBJECT OF THE PRESENT INVENTION

It is an object of the present invention to provide a simple, rapidly transdermally deployable topical preparation for the effective treatment of unwanted fat and lipodystrophies.

It is an object of the present invention to provide the consumer with a simple, non invasive, effective, rapidly deployable means and method for improving cosmetic appearance via the elimination of unwanted fat.

It is an object of the present invention to provide the consumer with a safe, simple and effective topical preparation to target the adipose tissue and to induce lysis of adipose cells in body areas of specific user's concern, exactly where those adipose cells aggregates are unwanted.

It is an object of the present invention to provide the consumer with a safe, simple, effective topical preparation such as cream, lotion, emulsion, paste, ointment and the likes having lipo-dissolving capabilities as substitute for lipo-dissolving intradermal injections which carry significantly more morbidity risks.

It is an object of the present invention to utilize a proven safe effective method of transdermal drug delivery such as the cyclodextrins technology for targeting specifically adipose tissue which is otherwise accessible only via injections.

It is another object of the present invention to utilize a safe and effective method of transdermal drug delivery based on transdermal permeability enhancers which promote accumulation in the subcutaneous tissue of the transdermally delivered substances, in combination with a negligible systemic absorption of such substances, to maximize in loco effect of lipo-dissolving substances on subcutaneous adipose tissue.

### BRIEF DESCRIPTION OF THE DRAWINGS

FIG. 1 shows a liposome vesicle in cross section.

FIG. 2 is a side, see thru view of a cyclodextrin.

### SPECIFICATIONS

The invention consists of a topical dermatological preparation containing a lipo-dissolving or adipocytes disrupting/lytic substance or ingredient or active principle or means associated with a skin penetration enhancer.

In one embodiment the lipo-dissolving or adipocyte disrupting/lytic ingredient is deoxycholic acid having the properties of an emulsifier/detergent/surfactant. Deoxycholic acid is a biliary compound. A large number of biliary compounds have been found, all having emulsifying/detergent/surfactant properties in various degrees, therefore all potentially effective in lipo-dissolving subcutaneous fat The list reported below includes a great number of the known biliary acid/salts compounds. Besides the deoxycholic acid or salt, other biliary compounds found in humans are the cholic acid or salt, chenodeoxycholic acid or salt and the lithocholic acid or salt.

Cholic Acids: 1,3,12-trihydroxycholanoic acid; 1,3,7,12-tetrahydroxycholanoic acid; 3beta-hydroxy-delta 5-cholenic acid; 3 beta-hydroxychol-3-en-24-oic acid; 3′-isothiocyanatobenzamidecholic acid; 3,12-dihydroxy-5-cholenoic acid; 3,4,7-trihydroxycholanoic acid; 3,6,12-trihydroxycholanoic acid; 3,7,12,23-tetrahydroxycholan-24-oic acid; 3,7,12-trihydroxy-7-methylcholanoic acid; 3,7,12-trihydroxycoprostanic acid; 3,7,23-trihydroxycholan-24-oic acid; 3,7-dihydroxy-22,23-methylene-cholan-24-oic acid (2-sulfoethyl)amide; 3-((3-cholamidopropyl)dimethylammonium)-1-propanesulfonate; 3-((3-deoxycholamidopropyl)diinethylammonio)-1-propane; 3-benzoylcholic acid; 3-hydroxy-5-cholen-24-oic acid 3-sulfate ester; 3-hydroxy-7-(hydroxyimino)cholanic acid; 3-iodocholic acid; 7,12-dihydroxy-3-(2-(glucopyranosyl)acetyl)cholan-24-oic acid; 7,12-dihydroxy-3-oxocholanic acid; allocholic acid; chapso; chol-3-en-24-oic acid; cholanic acid; Cholic Acid (which includes the Cholates: sodium cholate; methyl cholate; benzyldimethylhexadecylammonium cholate; methyl 1,3-dihydroxycholan-24-oate; and trioctylmethylammonium cholate); cholic acid glucuronide; cholyl-coenzyme A; cholyl-lysylfluorescein; cholyldiglycylhistamine; cholylhistamine; cholylhydroxamic acid; cholylsarcosine; cholyltetraglycylhistamine; ciliatocholic acid; Dehydrocholic Acid (which includes FZ 560; Gallo-Merz; Gillazym; Hepavis; Mexase; progresin Retard; and spasmocanulase); Deoxycholic Acid (which includes: 23-nordeoxycholic acid; 3,7-dioxocholanoic acid; 3-hydroxy-polydeoxycholic acid; 3-sulfodeoxycholic acid; 6-hydroxycholanoic acid; 6-methylmurideoxycholic acid; 7-ketodeoxycholic acid; 7-methyldeoxycholic acid; Chenodeoxycholic Acid; dehydrodeoxycholic acid; deoxycholyltyrosine; desoxybilianic acid; Glycodeoxycholic Acid; hyodeoxycholate-6-O-glucuronide; hyodeoxycholic acid; Taurodeoxycholic Acid; and Ursodeoxycholic Acid); Glycocholic Acid (which includes: 3-hydroxy-5-cholenoylglycine; cholylglycylhistamine; cholylglycyltyrosine; Glycodeoxycholic Acid; and sulfolithocholylglycine); hemulcholic acid; Lithocholic Acid (which includes: 12-ketolithocholic acid; 24-norlithocholic acid; 3-dehydrolithocholylglycine; 3-hydroxy-6-cholen-24-oic acid; 3-hydroxy-7,12-diketocholanoic acid; 3-hydroxy-7-methylcholanoic acid; 3-ketolithocholic acid; 3-oxochol-4-en-24-oic acid; 3-oxocholan-24-oic acid; 4-azidophenacy lithocholate; 7-ketolithocholic acid; BRL 39924A; glycolithocholic acid; lithocholate 3-O-glucuronide; lithocholyl-N-hydroxysuccinimide; methyl lithocholate; N-carbobenzoxy-N-lithocholyl-epsilon-lysine; N-epsilon-lithocholyllysine; sulfolithocholic acid; and Taurolithocholic Acid); muricholic acid; N-(1,3,7,12-tetrahydroxycholan-24-oyl)-2-aminopropionic acid; N-(2-aminoethyl)-3,7,12-trihydroxycholan-24-amide; N-carboxymethyl)N-(2-(bis(carboxymethyl)amino)ethyl)-3-(4-(N′-(2-((3,7,12-trihydroxycholan-24-oyl)amino)ethyl)(thioureido)phenyl)alanine; N-cholyl-2-fluoro-beta-alanine; norcholic acid; norursocholic acid; Taurocholic Acid (which includes: (N-(7-(nitrobenz-2-oxa-1,3-diazol-4-yl))-7-amino-3alpha,12alpha-dihydroxycholan-24-oyl)-2-arninoethanesulfonate; 23-seleno-25-homotaurocholic acid; 3,12-dihydroxy-7-oxocholanoyltaurine; 3-hydroxy-7-oxocholanoyltaurine; azidobenzamidotaurocholate; hexadecyltributylammonium taurocholate; tauro 1-hydroxycholic acid; tauro-3,7-dihydroxy-12-ketocholanoic acid; taurodehydrocholate; Taurodeoxycholic Acid; tauroglycocholic acid; Taurolithocholic Acid; tauromuricholic acid; tauronorcholic acid); tetrahydroxy-5-cholan-24-oic acid; ursocholic acid; vulpecholic acid; bile acid sulfates.

The Glycodeoxycholic Acid includes: Glycochenodeoxycholic Acid; 7-oxoglycochenodeoxycholic acid; glycochenodeoxycholate-3-sulfate; glycohyodeoxycholic acid; the Taurodeoxycholic Acid includes: tauro-7,12-dihydroxycholanic acid; Taurochenodeoxycholic Acid; taurochenodeoxycholate-3-sulfate; taurochenodeoxycholate-7-sulfate; tauroursodeoxycholic acid; taurohyodeoxycholic acid; the Ursodeoxycholic Acid includes: 23-methylursodeoxycholic acid; 24-norursodeoxycholic acid; 3,6-dihydroxy-6-methylcholanoic acid; 3,7-dihydroxy-20,22-methylenecholan-23-oic acid; 3,7-dihydroxy-22,23-methylenecholan-24-oic acid; 3,7-dihydroxy-7-ethylcholanoic acid; 3,7-dihydroxy-7-methylcholanoic acid; 3,7-dihydroxy-7-n-propylcholanoic acid; Bamet-UD2; diamminebis(ursodeoxycholate(O,O′))platinum(II); glycoursodeoxycholic acid; homoursodeoxycholic acid; HS 1030; HS 1183; isoursodeoxycholic acid; PABA-ursodeoxycholic acid; sarcosylsarcoursodeoxycholic acid; sarcoursodeoxycholic acid; ursodeoxycholate-3-sulfate; ursodeoxycholic acid 7-oleyl ester; ursodeoxycholic acid N-acetylglucosaminide; ursodeoxycholic acid-3-O-glucuronide; ursodeoxycholyl N-carboxymethylglycine; ursodeoxycholylcysteic acid; Ursometh; the Chenodeoxycholic Acid includes: 24-norchenodeoxycholic acid; 3,7-dihydroxy-12-oxocholanoic acid; 3,7-dihydroxy-24-norcholane-23-sulfonate; 3,7-dihydroxy-25-homocholane-25-sulfonate; 3,7-dihydroxychol-5-enoic acid; 3,7-dihydroxycholane-24-sulfonate; 3-glucosido-chenodeoxycholic acid; 3-oxo-7-hydroxychol-4-enoic acid; 6-ethylchenodeoxycholic acid; chenodeoxycholate sulfate conjugate; chenodeoxycholyltyrosine; Glycochenodeoxycholic Acid which includes: 7-oxoglycochenodeoxycholic acid and glycochenodeoxycholate-3-sulfate; homochenodeoxycholic acid; HS 1200; methyl 3,7-dihydroxychol-4-en-24-oate; methyl 3,7-dihydroxycholanate; N-(2-aminoethyl)-3,7-dihydroxycholan-24-amide; N-chenodeoxycholyl-2-fluoro-beta-alanine; sarcochenodeoxycholic acid; Taurochenodeoxycholic Acid; taurochenodeoxycholate-3-sulfate;taurochenodeoxycholate-7-sulfate; tauroursodeoxycholic acid.

The above list is by all means not complete. It is only reported to mention instances of the class of biliary compounds, either natural as they occur in different species or synthetic or semi-synthetic.

Other emulsifier/detergent/surfactants having lipo-dissolving or adipocytes disrupting/lytic properties can be used, such as a nonionic surfactant, as octyl phenol ethoxylate, also known as polyoxyethylene or octyl phenyl ether, or a cationic surfactant, such as cetyltrimethylammonium bromide, or mixed nonionic and anionic surfactants, or zwitterionic surfactants, such as N-Dodecyl-N,N dimethylglycine.

In another embodiment the lipo-issolving or adipocytes disrupting ingredient is a metalloproteinase enzyme having lipo-issolving or adipocytes disrupting properties, such as a collagenase. Other enzymes can be used such as Bromelase/Bromelin, Lipase, Papaine.

In yet another embodiment the lipo-issolving or adipocyte disrupting ingredient is Phosphatidylcholine which also has lipo-dissolving or adipocytes disrupting/lytic properties.

Other embodiments contain a combination of either two or more of the lipo-issolving or adipocytes disrupting/lytic substances described above.

Along with the lipo-issolving or adipocyte disrupting, substance or substances, either used alone or in combination, the topical preparation contains a dermatological vehicle/excipient proper of the preparation, vehicle /excipient that confers to the dermatological preparation the physical/chemical properties of a cream, lotion, gel, paste, or the physical/chemical properties of wet preparations such as bath, soaks wet dressings, skin patches and the likes. To mention just a few: lanoline, starch, glycerin, petrolatum, paraffin wax.

In each of the above embodiments, skin penetration enhancers which facilitate transport of the active principles thru the skin into the target tissue are added to the dermatological preparation. or are used as external physical facilitators.

The skin permeability enhancers that can be used include Percutaneous Chemical Enhancers and Percutaneous Physical Enhancers.

The Percutaneous Chemical Enhancers which are added to the lipo-dissolving compound can be classified as: Cyclodextrins, Liposomes, Ethosomes, Sulfoxides, Alcohols, Fatty acids, Fatty acid esters, Polyols, Amides Surfactants, Terpene, Alkanones Organic acids.

Specifically Percutaneous Chemical Enhancers which can be used are: Ethanol, Glyceryl monoethyl ether, Monoglycerides, Isopropylmyristate, Lauryl alcohol (also, lauric acid, lauryl lactate), Terpinol, Menthol, D-limonene, Beta-cyclodextrin, DMSO (dimethyl sulfoxide), Polysorbates, Fatty acids e.g. oleic, N-methylpyrrolidone, Polyglycosylated glycerides, 1-Dodecylaza cycloheptan-2-one known as Azone®, Cyclopentadecalactone known as CPE-215®, Alkyl-2-(N,N-disubstituted amino)-alkanoate ester, known as NexAct®, 2-(n-nonyl)-1,3-dioxolane known as SEPA®.

Generally the purpose of transdermal delivery of a therapeutic substance is to reach the systemic circulation for distribution of the substance by the blood.

The purpose for trasdermal delivery subject of the present application is to deliver most of the lipo-dissolving substance, namely an emulsifier, locally into the adipose subcutaneous tissue maximizing subcutaneous accumulation while minimizing systemic absorption in order to achieve maximization of local effect.

Indeed, in most cases, the dermal microvasculature absorbs the drug being delivered placing it into the systemic circulation before it ever reaches the adipose tissue.

A study conducted at USC, “Delivery of erythromycin to subcutaneous tissues in rats by means of a trans-phase delivery system” by Peng L, Nimni ME, J Pharm Pharmacol. 1999 October; 51(10):1135-41, has confirmed that a mixture of Benzyl Alcohol, Acetone and Isopropanol being used as trasdermal permeability enhancer has resulted with significant and persistent local accumulation in the subcutaneous tissue of the substance being transdermally delivered while a standard hydrophilic transdermal carrier such as propylene glycol has shown a good systemic absorption of the substance being transdermally delivered, but a very negligible and short living subcutaneous accumulation of the substance in the subcutaneous tissue.

Applicants suggest that such mixture of Benzyl Alcohol, Acetone and Isopropanol satisfies the above condition of transporting the therapeutic substance across the epidermal barrier and promises significant and persistent accumulation of the therapeutic substance in the adipose tissue minimizing systemic absorption.

Such a formulation has the potential for achieving high local tissue concentration the in vicinity of the site of application, and, consequently, it has the potential for maximizing local pharmacological effect of lipo-dissolving substances on subcutaneous adipose tissue in the proximity of the site of application. The respective concentration of Benzyl Alcohol, Acetone and Isopropanol varies, however the recommended concentration is: benzyl alcohol (10%), acetone (40%), isopropanol (50%).

In addition to chemical transdermal permeability enhancers, physical transcermal enhancers can be used. Physical Enhancers which can be used are Iontophoresis, Electroporation, Sonophoresis, Thermal Poration and in general physically/chemically induced heat, Microneedles, Dermabrasion.

Topical delivery of subcutaneous tissues lipo-dissolving substances can also be enhanced after microdermabrasion. Microdermabrasion can be used alone or in combination with Percutaneous Chemical Enhancers, such as the Percutaneous Chemical Enhancers described above and/or with Physical Enhancers, such as the Physical Enhancers described above.

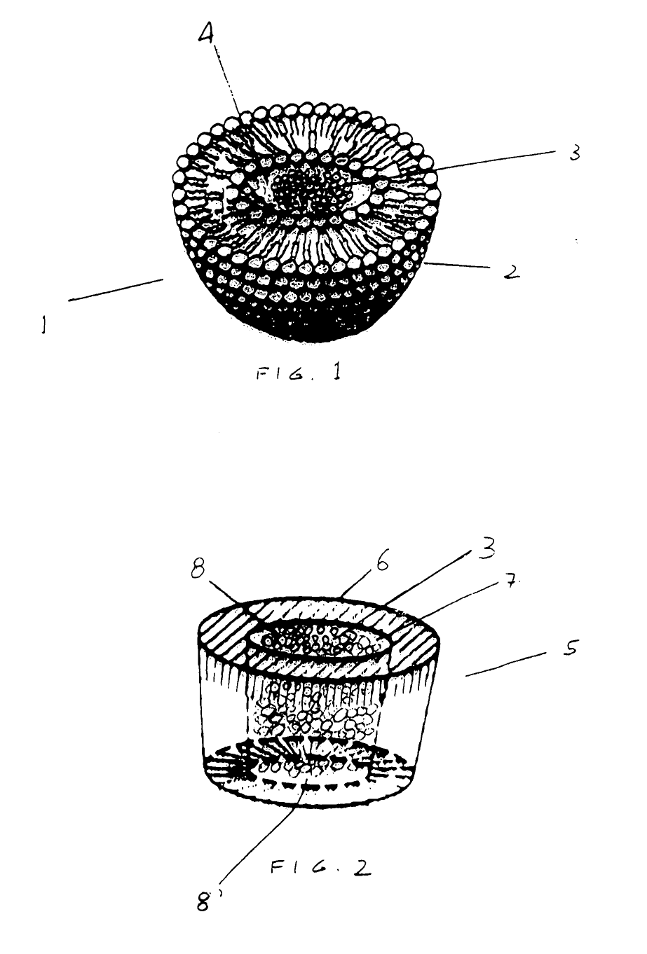

FIG. 1 shows liposome vesicle 1 in cross section while FIG. 2 is a side view of cyclodextrin 5.

As shown in FIG. 1, liposome vesicle 1 is of spherical shape with multilayer phospholipids membrane wall 2 delimiting inner cavity 4 for payload 3.

Payload 3 can be any of the above mentioned lipo-dissolving substances.

FIG. 2 shows cyclodextrin unit 5 , generally of toroid shape having polysaccharide wall 6 delimiting cavity 7 for payload 3. Toroid shaped cyclodextrin 5 is formed with upper larger opening 8 and lower smaller opening 8A. Payload 3 is a lipo-dissolving compound preferably a biliary compound, as listed above.

Lipodissolving substances alone or in combination delivered by topical dermatological preparations into the skin will diffuse according different factor of penetration into the dermis and into the subcutaneous tissues targeting specifically the adipose tissue. Lipo-dissolving substances alone or in combination will dissolve, induce lysis of the adipose tissue. Adipocytes cell wall will be disrupted and its content will be lysed. Lipo-dissolving substances such as Deoxycholic acid have indeed repeatedly shown in studies to have the property and capability of dissolving fat cell aggregates once in contact with such cells, as shown by a study conducted at UCLA by A. Rotunda et al. “Detergent effects of sodium deoxycholate are a major feature of an injectable phosphatidylcholine formulation used for localized fat dissolution”, Dermatological Surg. 2004 July; 30(7):1001-8, and by another UCLA study by A.Rotunda et al. “Lipomas treated with subcutaneous deoxycholate injections”, J Am Acad Dermatol. 2005 December; 53(6):973-8.

The transdermal administration through topical dermatological preparations will offer a number of advantages over parenteral administration via injections, including ease of administration, elimination of puncturing discomforts, reduction of possibility of infections, convenience of treatments, a more homogeneous distribution of the active substance into the target organ than possible via injections with more satisfactory aesthetical results, reduced formation of lumps in the subcutaneous areas being treated, and avoidance of hematomas formation.

What we claim is:

1. A topical dermatological preparation for subcutaneous fat tissue treatment comprising:
a lipo-dissolving emulsifier, and
a cyclodextrin permeability enhancer, wherein said cyclodextrin permeability enhancer enhances skin permeability to said lipo-issolving emulsifier.

2. The topical dermatological preparation for subcutaneous fat tissue treatment of claim 1 wherein said lipo-dissolving emulsifier is a biliary compound.

3. The topical dermatological preparation for subcutaneous fat tissue treatment of claim 2 wherein said lipo-dissolving emulsifier is deoxycholic acid.

4. A topical dermatological preparation for subcutaneous fat tissue treatment comprising:
a lipo-dissolving emulsifier, and
a skin permeability enhancer, wherein said skin permeability enhancer enhances skin permeability to said emulsifier and maximizes local effect of said emulsifier on subcutaneous fat tissue by maximizing accumulation of said emulsifier into the subcutaneous fat tissue while minimizing systemic absorption.

5. The topical dermatological preparation for subcutaneous fat tissue treatment of claim 4 wherein said skin permeability enhancer is a mixture of Benzyl Alcohol, Acetone and Isopropanol.

6. The topical dermatological preparation for subcutaneous fat tissue treatment of claim 4 wherein said lipo-issolving emulsifier is a biliary compound.

7. The topical dermatological preparation for subcutaneous fat tissue treatment of claim 6 wherein said skin permeability enhancer is a mixture of Benzyl Alcohol, Acetone and Isopropanol.

8. The topical dermatological preparation for subcutaneous fat tissue treatment of claim 4 wherein said lipo-dissolving emulsifier is deoxycholic acid.

9. The topical dermatological preparation for subcutaneous fat tissue treatment of claim 6 wherein said skin permeability enhancer is a mixture of Benzyl Alcohol, Acetone and Isopropanol.

10. The topical dermatological preparation for subcutaneous fat tissue treatment of claim 4 wherein said lipo-dissolving emulsifier is a salt of deoxycholic acid.

11. The topical dermatological preparation for subcutaneous fat tissue treatment of claim 6 wherein said skin permeability enhancer is a mixture of Benzyl Alcohol, Acetone and Isopropanol.

12. A topical dermatological preparation for subcutaneous fat tissue treatment comprising:
a lipo-issolving substance, and
a cyclodextrin permeability enhancer, wherein said cyclodextrin permeability enhancer enhances skin permeability to said lipo-issolving substance.
